(19) United States
(12) Patent Application Publication
(10) Pub. No.: US 2004/0180017 A1
(43) Pub. Date: Sep. 16, 2004

(54) Cosmetic product

(76) Inventors: Mark Constantine, Dorset (GB); Margaret Constantine, Dorset (GB); Helen Ambrosen, Dorset (GB); Joanne Evans, Dorset (GB)

Correspondence Address: Westman Champlin & Kelly, International Centre Suite 1600, 900 Second Avenue South, Minneapolis, MN, 55402-3319, US

(21) Appl. No.: 10/332,109

(22) PCT Filed: Jul. 4, 2001
(86) PCT No.: PCT/GB01/03007

(30) Foreign Application Priority Data
Jul. 6, 2000 (GB) 00166660

(51) Int. Cl. A61K 7/06; A61K 7/11; A61K 7/075; A61K 7/08

(52) U.S. Cl. 424/70.11; 424/70.21

(57) ABSTRACT

A cosmetic product comprising a surfactant and water packaged as several individual applications of the product in packaging enabling the product to be frozen and used in solid frozen form. The cosmetic product preferably comprises, by weight; 5% to 15% amphoteric surfactant and 30% to 40% other surfactant. The use of a frozen gel when showering is highly invigorating and, at least gives the sensation of, stimulating the blood circulation through a systemic effect on the body. It also enables a predefined quantity of surfactant to be applied over the body in a controlled manner, thereby minimising potential harmful effects arising from the application of excessive amounts of surfactant to areas of the body.

The present invention relates to cosmetic products such as shower gels and shampoos.

The invention has been made with a view to providing a basis for a new product which is invigorating to use and which may also provide other benefits.

According to a first aspect of the present invention there is provided a cosmetic product comprising a surfactant and water, and arranged in packaging comprising a plurality of chambers, each containing an individual application of the cosmetic product, and wherein the packaging is suitable for enabling the cosmetic product to be frozen and used as individual applications of the cosmetic product in solid frozen form.

Advantageously the cosmetic product comprises, by weight; 5% to 15% amphoteric surfactant and 30% to 40% other surfactant.

In a most preferred embodiment of the invention the cosmetic product is frozen

According to a second aspect of the present invention there is also provided a method of applying a cosmetic product according to the first aspect to the skin of a human body, the method comprising freezing the cosmetic product thereby to provide the cosmetic product in a frozen solid form, thereby to facilitate the application of the cosmetic product over the surface of the skin of the human body.

Embodiments of the present invention will now be described, by way of further example only, with reference to the accompanying drawings in which:

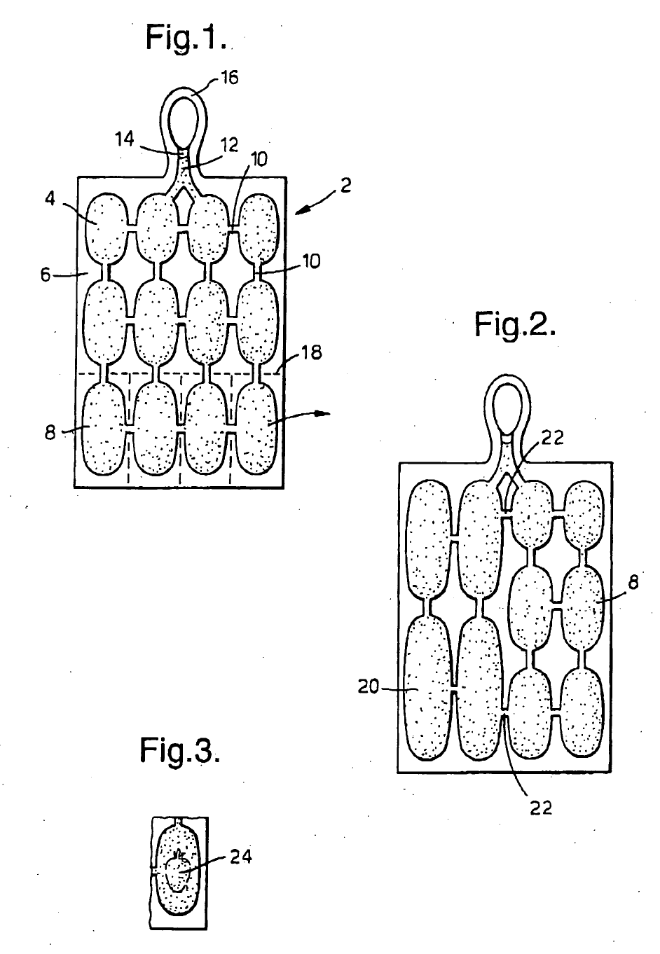

FIG. 1 shows a cosmetic product in accordance with an embodiment of the present invention;

FIG. 2 shows a cosmetic product in accordance with a further embodiment of the present invention; and

FIG. 3 shows an individual application of the cosmetic product.

It has been realised with the present invention that the use of a frozen gel when showering is highly invigorating and, at least gives the sensation of, stimulating the blood circulation. This arises because the application of the frozen gel to the skin provides a systemic reaction within the body and not just a reaction in that part of the body immediately underlying that area of the sin to which the frozen gel is applied.

Although with hindsight the freezing of a shower gel so as to provide a product invigorating to use may seem simple, apart from not having been proposed previously it also has to be recognised that the simple freezing of a conventional shower gel is not considered appropriate. Thus, in addition to recognition of the unexpected invigorating effect of using a frozen shower gel in a warm water environment, the invention is also predicated on the recognition that simply freezing a conventional gel is most likely to result in the application of too large a quantity of surfactant material to the skin and/or hair.

For ease of use, a solid block of frozen shower gel should desirably be up to the size of a conventional bar of soap, e.g. 10 cm×6 cm×4 cm or something of that order. Typically such a block might therefore contain up to about 100 grams of gel, which is about half that of a conventional 200 gram bottle of shower gel. The use of such a large quantity of conventional gel in one application could be expected to have a potentially undesirable effect on the skin, as well as being relatively expensive use of the product compared with the number of applications that would normally be obtained from such a quantity of gel. Thus an embodiment of the present invention resides in a shower gel of sufficiently reduced concentration to provide a desirable amount of active ingredients when frozen in a block up to the aforementioned size.

Shower gel products have existed for many years. They are useful multipurpose products which allow a user to wash the hair and body whilst under the running water of a shower. Many people also use them as a way of cleansing the hair and skin in the bath. They are usually applied to the hair by applying the product onto the hand then massaging in or squeezing the product directly from its container onto the head. On the skin they can be applied directly or squeezed onto a sponge, then massaged to lather into a foam on the skin.

Because shower gel formulations are not only applied to the hair but also the skin, they need to be capable of de-greasing the hair but must also be mild enough not to irritate the skin. However, because the gel is in liquid form, and because in a shower the gel is being applied in the water stream from the shower head, it is difficult to spread a film of the liquid shower gel over sufficient area of the body using a small quantity of the gel. Generally, a large proportion of the gel held on the hand is deposited onto that area of the body first contacted by the hand, and the remaining gel held on the hand is then spread to other parts of the body. For this reason, many users of such gels tend to apply the gel in a piecemeal fashion, dispensing gel from the container several times during the course of a shower to ensure that the body is adequately covered and therefore cleansed. However, as will be appreciated, such application of the shower gel to the body means that certain areas of the body will be exposed to too large a quantity of surfactant material, with potentially undesirable effects on the skin, whilst other areas of the body may receive too little surfactant material, giving rise to minimal cleansing from the gel. Additionally, a significant proportion of the gel may be washed from the hand in the water stream before there is any cleansing effect to the skin. Hence, a significant amount of the shower gel may be washed to waste down the shower drain unused. Certain liquid shower gels have been sold in containers with dispensers at the outlet to ensure a measured quantity of gel is made available to a user but in the running water stream it is difficult to spread such relatively small quantities of liquid gel over the body. Many users, therefore, turn off the water stream whilst the shower gel is applied and then turn back on the water stream to subsequently lather the gel and wash the body. However, this can also be very inconvenient, particularly as a significant proportion of showers are not thermostatically controlled, requiring the user to readjust the hot and cold water flows whilst in the shower in order to provide the correct water temperature for washing with the gel.

Thus, it has been realised with the present invention, that if the shower gel is frozen into a solid form, the application of the surfactant in the gel can be applied more easily to all areas of the body in the warm water stream. Furthermore, as the gel is frozen in a quantity sufficient to ensure that the gel can be applied over the skin area, excess gel is not used and more even application of the gel, and thus surfactant, over the body can be achieved, with the result that the potentially undesirable effects of applying an excess of surfactant to certain areas of the skin can be minimised. Additionally, there is the added benefit that a milder formulation for the shower gel can be efficiently used, again contributing to minimising the undesirable effects on the skin. Moreover, the ability to simultaneously apply the frozen gel and the warm water to the skin helps to invigorate the skin, assisting in the cleansing process. These benefits are in addition to the greater than expected invigorating effect arising from using a frozen block of gel in the warm water environment which, as stated above, induces a deep systemic reaction within the body.

Shower gels are usually available in plastic tubes or bottles. These containers allow the product to be applied easily and will not break in the bath or shower. However, they are often not biodegradable and, as they are produced in a relatively rigid material to ensure that they return to shape after squeezing to enable subsequent dispensing of the gel, produce relatively bulky waste.

Blocks of frozen shower gel according to the present invention can be packaged in paper, cardboard or very thin plastics sheet material, such as polythene, thus providing the benefit of more environmentally friendly packaging compared with conventionally packaged conventional shower gels.

FIG. 1 shows a cosmetic product in the form of a shower gel in accordance with the present invention. The cosmetic product 2 comprises a shower gel 4 contained in packaging 6 which is made from two sheets of thin plastics material, such as polythene, bonded together by any suitable process, such as by heating. The sheets of polythene material, which are not discernible from FIG. 1, are bonded in such a way as to produce chambers 8 with walls of the thin plastics material. The chambers 8 shown in FIG. 1 are formed as rows and columns and conduits 10 are provided between adjacent chambers to facilitate filling of the chambers with the cosmetic product. The packaging 6 also includes a further conduit 12 through which the chambers 8 may be filled. The further conduit 12 may also be provided with a simple one-way valve arrangement 14 to allow the gel to flow into the packaging but to ensure the gel does not subsequently leak from the packaging. A loop member 16 is also provided which may conveniently be used to carry the cosmetic product or to hang the cosmetic product from a suitable support, such as a hook or pole.

The cosmetic product in the form of a liquid shower gel is fed into the container via the further conduit 12 and the liquid gel flows through the chambers 8 and 10 until all of the chambers are filled with the liquid gel. The walls of the chambers are formed by the thin plastics material, which is relatively resilient. Hence, when the packaging 6 filled with liquid gel is subsequently frozen, the walls of the chambers 8 can flex and expand to accommodate the increase in volume of the liquid gel caused by freezing without rupturing of the plastics sheet. This means that the containers 6 can be completely filled with the gel in liquid form. This is particularly advantageous as the packaging 6 is preferably made from a substantially transparent plastics material and the liquid gel can be provided as a variety of coloured liquid formulations, depending on the chosen ingredients. The chambers 8 filled with the coloured liquid gel can therefore provide a good contrast to the background areas of the transparent material, providing enhanced attractiveness at a point of sale, with the cosmetic product being suspended from a suitable support by the loop member 16. As will be appreciated, packagings filled with the liquid gel in a variety of colours may then be attractively displayed at a point of sale.

The chambers 8 may be of any suitable size but, preferably, they are each sized to accommodate sufficient quantity of shower gel for use as an individual application in a shower. However, some users may require to apply a larger quantity of shower gel compared to other users. Hence, it is also possible to provide the cosmetic product with packaging having chambers of different size, such as is shown in FIG. 2, where larger chambers 20 are also provided. In this manner, standard and larger individual applications of the cosmetic product can be provided for in common packaging. Furthermore, as will be appreciated, although four larger individual applications and six standard individual applications are shown in FIG. 2, the same number of larger and standard individual applications can be provided in the common packaging. Also, the larger individual applications may be provided in a different fragrance to that of the standard individual applications to more closely align the respective applications to the preferences of the potential male and female users. This would necessitate the provision of individual fill conduits for the larger and standard individual applications and also the deletion of interconnect conduits 22 between the standard and larger chambers.

The packaging may also be provided with a patterned surface deformation, such as the symbol 24 shown in FIG. 3, which can be used to indicate to a user by sight and or feel, a characteristic of the cosmetic product, such as the fragrance or a particular ingredient in the composition of the cosmetic product, such as a fruit from which a fruit juice additive is derived.

In use, the cosmetic product illustrated in FIG. 1 is placed in a freezer until the liquid gel is frozen into solid frozen form. A chamber 8 containing an individual application of the cosmetic product in solid frozen form may then be removed or separated from the packaging, such as by cutting or tearing along lines 18, as shown in FIG. 1, to provide an individual application of the cosmetic product, as shown in FIG. 3. The frozen cosmetic product can then be easily removed from the packaging and used in the shower, with the attendant advantages of being able to apply a milder surfactant formulation over the body whilst providing systemic stimulation to the body. Hence, a particularly invigorating shower can be provided whilst at the same time minimising any detrimental effects to the skin through potential uneven or over application of stronger surfactant formulations.

The frozen shower gel is used directly onto the skin in the warm water of the shower (or bath if desired). The combination of the cold shower gel and the warm water stimulates the circulation. The result is an effect much enhanced by the action of the frozen shower gel. The muscles of the body are revived and when used on the head the effect refreshes the senses. These effects are enhanced not only by the use of a ‘cold’ product, but also by the fact that the product is used in solid form, enabling the frozen solid block to be used with a rubbing action over the skin with the simultaneous application of warm water. Alternatively, the cosmetic product may be applied over the skin with subsequent application of the warm water.

An example of a formulation for a shower gel according to an embodiment of the present invention is as follows:

| | % |
|---|---|
| Surfactant e.g. Sodium Laureth Sulphate | 35.0 |
| Amphoteric Surfactant e.g. Disodium Cocoamphodiacetate | 8.0 |
| Foam Booster e.g. Lauryl Betaine | 1.5 |
| Fragrance | 0.7 |
| Water and/or other additives e.g. fruit juices, herbal infusions | 54.5 |
| Preservative e.g. Methyl Paraben and Propyl Paraben | 0.3 |
| | 100.0 |

In contrast to the example given above, an example of a formulation for a conventional shower gel is as follows:

| | % |
|---|---|
| Surfactant e.g. Sodium Laureth Sulphate | 45.0 |
| Amphoteric Surfactant e.g. Disodium Cocoamphodiacetate | 20.0 |
| Foam Booster e.g. Lauryl Betaine | 1.0 |
| Fragrance | 0.7 |
| Water and/or other additives e.g. fruit juices, herbal infusions | 33.0 |
| Preservative e.g. Methyl and Propyl Paraben | 0.3 |
| | 100.0 |

Clearly, the quantity of surfactant provided by one individual application (i.e. one frozen block of gel according to the invention) must be sufficient to provide a desirable level of cleansing effect whilst avoiding too high a concentration, as discussed above. A sufficient quantity of bubbles or lather is also provided by the formulation. This effect is felt to be essential by the users who would not preferably use a non or low lathering product. Thus, it has been determined that a cosmetic product according to the present invention should comprise, by weight; 5% to 15% amphoteric surfactant and 30% to 40% other surfactant. As already noted, preferably the remainder is mainly water. Such a formulation has a relatively low viscosity. Hence, if used in liquid form, a thickening agent would almost certainly be required. Freezing of the cosmetic product provides, therefore, the additional benefit of being able to use such a mild formulation without the use of thickening agents.

Preferred formulations according to the present invention are indicated by the following percentage ranges of typical ingredients, namely:

| | % |
|---|---|
| Surfactant e.g. Sodium Laureth Sulphate | 30.0-40.0 |
| Amphoteric Surfactant e.g. Disodium Cocoamphodiacetate | 5.0-15.0 |
| Foam Booster e.g. Lauryl Betaine | 2.5-1.5 |
| Fragrance | 0.7-0.7 |
| Water and/or other additives e.g. fruit juices, herbal | 61.5-42.5 |
| infusions | |
| Preservative e.g. Methyl and Propyl Paraben | 0.3-0.3 |
| | 100.0 100.0 |

This invention might also be applied to shampoos, hair treatments such as conditioners or gels, soap formulations, aftersun and massage products where by using a frozen formula, stimulation of the circulation and refreshing effects are experienced.

1. A cosmetic product comprising a surfactant and water, and arranged in packaging comprising a plurality of chambers, each containing an individual application of the cosmetic product, and wherein the packaging is suitable for enabling the cosmetic product to be frozen and used as individual applications of the cosmetic product in solid frozen form.

2. A cosmetic product as claimed in claim 1, wherein the packaging comprises first and further sheets of a substantially transparent material bonded together to provide the chambers therebetween and for enabling the cosmetic product to be viewed in the packaging.

3. A cosmetic product as claimed in claim 2, wherein the substantially transparent material comprises polythene.

4. A cosmetic product as claimed in any one of claims 1 to 3, wherein the chambers are arranged as an array of columns, each column comprising a plurality of the chambers, and comprising conduits interconnecting adjacent chambers in a column for facilitating filling of the chambers with the individual applications of the cosmetic product.

5. A cosmetic product as claimed in any one of claims 1 to 4 comprising chambers of different sizes for providing respectively first and further quantities of the cosmetic product as individual applications in common packaging for the cosmetic product.

6. A cosmetic product as claimed in claim 5, wherein the first quantity of cosmetic product is provided with a first fragrance which differs from a further fragrance provided for the further quantities of the cosmetic product.

7. A cosmetic product as claimed in any one of claims 1 to 6, wherein the packaging comprises a loop member for enabling the cosmetic product to be hung from a support member.

8. A cosmetic product as claimed in any one of claims 1 to 7 comprising, by weight; 5% to 15% amphoteric surfactant and 30% to 40% other surfactant.

9. A cosmetic product as claimed in claim 8, wherein the amphoteric surfactant comprises disodium cocoamphodiacetate.

10. A cosmetic product as claimed in claim 8 or claim 9, wherein the said other surfactant comprises sodium laureth sulphate.

11. A cosmetic product as claimed in any one of claims 1 to 10, comprising by weight 42.5% to 61.5% water.

12. A cosmetic product as claimed in any one of the preceding claims, further comprising by weight 2.5% to 1.5% of foam booster.

13. A cosmetic product as claimed in claim 12, wherein the foam booster comprises lauryl betaine.

14. A cosmetic product as claimed in any one of the preceding claims, further comprising by weight 0.7% fragrance.

15. A cosmetic product as claimed in any one of the preceding claims, further comprising by weight 0.3% preservatives.

16. A cosmetic product as claimed in claim 15, wherein the preservative comprises methyl and propyl paraben.

17. A cosmetic product as claimed in any one of the preceding claims, further comprising a fruit juice additive.

18. A cosmetic product as claimed in any one of the preceding claims, further comprising a herbal infusion additive.

19. A cosmetic product as claimed in claim 6 or claim 13, wherein the packaging comprises a surface deformation for indicating by sight and/or feel to a user the fragrance of the cosmetic product.

20. A cosmetic product as claimed in claim 17, wherein the packaging comprises a surface deformation for indicating by sight and/or feel to a user a fruit from which the fruit juice additive is derived.

21. A cosmetic product as claimed in any one of claims 1 to 7 comprising an aftersun or massage product.

22. A cosmetic product as claimed in any one of claims 1 to 20 comprising a hair treatment product.

23. A cosmetic product as claimed in any one of claims 1 to 20 comprising a soap product.

24. A cosmetic product as claimed in any one of the preceding claims wherein the cosmetic product is in frozen form.

25. A method of applying a cosmetic product as claimed in any one of claims 1 to 23 to the skin of a human body, the method comprising freezing the cosmetic product thereby to provide the cosmetic product in a frozen solid form to facilitate the application of the cosmetic product over the surface of the skin of the human body.
